(19) United States
(12) Patent Application Publication
(10) Pub. No.: US 2008/0228145 A1
(43) Pub. Date: Sep. 18, 2008

(54) GUIDEWIRE INTRODUCER AND SHAPING TOOL

(75) Inventor: David A. Watson, San Jose, CA (US)

Correspondence Address: FULWIDER PATTON LLP, HOWARD HUGHES CENTER, 6060 CENTER DRIVE, TENTH FLOOR, LOS ANGELES, CA, 90045, US

(73) Assignee: Micrus Endovascular Corporation, San Jose, CA (US)

(21) Appl. No.: 12/049,167

(22) Filed: Mar. 14, 2008

Related U.S. Application Data
(60) Provisional application No. 60/918,128, filed on Mar. 15, 2007.

(51) Int. Cl. A61M 25/09

(52) U.S. Cl. 604/164.13

(57) ABSTRACT

The improved guidewire introducer and shaping tool includes a polymer inner layer and a metal outer layer. A polymer luer hub is provided on a polymer tubing shaft so that the internal taper of the hub has a smooth transition to the inner diameter of the polymer tubing shaft. A metal outer layer is provided over the outer surface of the polymer layer and can be rigidly attached to the hub to provide support and stiffness to the polymer layer.

### CROSS-REFERENCES TO RELATED APPLICATIONS

This application is based upon U.S. Provisional Application No. 60/918,128, filed 15 Mar. 2007, which is incorporated by reference in its entirety.

### BACKGROUND OF THE INVENTION

This invention relates generally to guidewire introducers for inserting a guidewire into a microcatheter for interventional therapeutic treatment or vascular surgery, and more particularly concerns a guidewire introducer having a polymeric inner tubular layer and a metal outer tubular layer. The guidewire introducer can be used for introduction of a wide variety of guidewires and vasoocclusive devices into microcatheters.

Introducers are commonly used to advance guidewires into a hub of a microcatheter, and are also commonly used as shaping mandrels to impart a curved shape to a tip of a guidewire. One common type of introducer that is currently available is constructed by insert molding a plastic luer hub onto a stainless steel hypodermic cannula or hypotube, and resembles a large bore, blunt ended injection needle. Since guidewires typically are coated with a polymer coating, such as polytetrafluoroethylene (PTFE), for example, this type of introducer has the potential to damage a guidewire during insertion of a guidewire through the introducer, but more commonly may damage a guidewire if the guidewire is withdrawn or even partially retracted back through the introducer. If the guidewire contacts the sharp end of the hypotube, skiving of the polymer coating of the guidewire can occur. However problematic insertion and retraction of guidewires through this type of introducer may be, these metal introducers make excellent shaping tools due to their stiffness and small outer diameters.

Some introducer manufacturers avoid the skiving issue by making an introducer from extruded polymer tubing rather than from stainless steel hypotube. One such type of introducer with a deflectable tip is formed from polyethylene. However, such polymeric introducers typically are not as stiff as metal introducers, and typically have larger outer diameters than metal introducers, since the polymer tubing typically needs to be relatively thicker walled than metal tubing to provide sufficient rigidity, and such polymeric introducers typically are therefore not as useful as shaping tools.

It would be desirable to provide a guidewire introducer that combines the benefits of both metal introducers and polymeric introducers into a guidewire introducer that is useful and less problematic for inserting and withdrawing or retracting a guidewire through a microcatheter, and that is also useful as a shaping tool. The present invention meets these and other needs.

### SUMMARY OF THE INVENTION

Briefly, and in general terms, the improved guidewire introducer and shaping tool includes a polymer inner layer and a metal outer layer. A polymer luer hub is provided on a polymer tubing shaft so that the internal taper of the hub has a smooth transition to the inner diameter of the polymer tubing shaft. A metal outer layer is provided over the outer surface of the polymer layer and can be rigidly attached to the hub to provide support and stiffness to the polymer layer.

Accordingly, the present invention provides for a guidewire introducer and shaping tool for placement of guidewires within a patient's vasculature, including an inner tubular shaft formed of polymeric material, and a tubular metal outer layer surrounding and extending over at least a portion of the inner tubular shaft from the proximal end of the inner tubular shaft. In a broad aspect, the inner tubular shaft may extend beyond the distal end of the tubular metal outer layer. In another presently preferred aspect, the inner tubular shaft includes a luer hub formed of polymeric material at the proximal end of the inner tubular shaft, and the luer hub includes a hub inner lumen connected with the inner guidewire lumen of the inner tubular shaft. In a presently preferred aspect, the hub inner lumen includes a surface defining an internal taper forming a smooth transition to the inner guidewire lumen of the inner tubular shaft. The proximal end of the tubular metal outer layer is preferably rigidly attached to the luer hub such that the tubular metal outer layer provides support and stiffness to the inner tubular shaft. In addition, the luer hub may have a distal external face, and an outer polymeric tubular sheath may be removably fitted onto the distal external face of the luer hub and over the tubular metal outer layer, with the outer polymeric tubular sheath extending beyond the distal ends of the inner tubular shaft and the tubular metal outer layer to protect the guidewire introducer until it is ready for use.

These and other aspects and advantages of the invention will become apparent from the following detailed description and the accompanying drawings, which illustrate by way of example the features of the invention.

### BRIEF DESCRIPTION OF THE DRAWINGS

FIG. 1 is a top plan view of the improved guidewire introducer and shaping tool according to the invention.

FIG. 2 is a schematic, cross-sectional view of the guidewire introducer and shaping tool of FIG. 1.

FIG. 3 is an end view at the proximal luer hub end of the guidewire introducer and shaping tool of FIG. 1.

FIG. 4 is an end view at the distal end of the guidewire introducer and shaping tool of FIG. 1.

FIG. 5 is a top plan view of the improved guidewire introducer and shaping tool of FIG. 1 with a removable protective sheath in place.

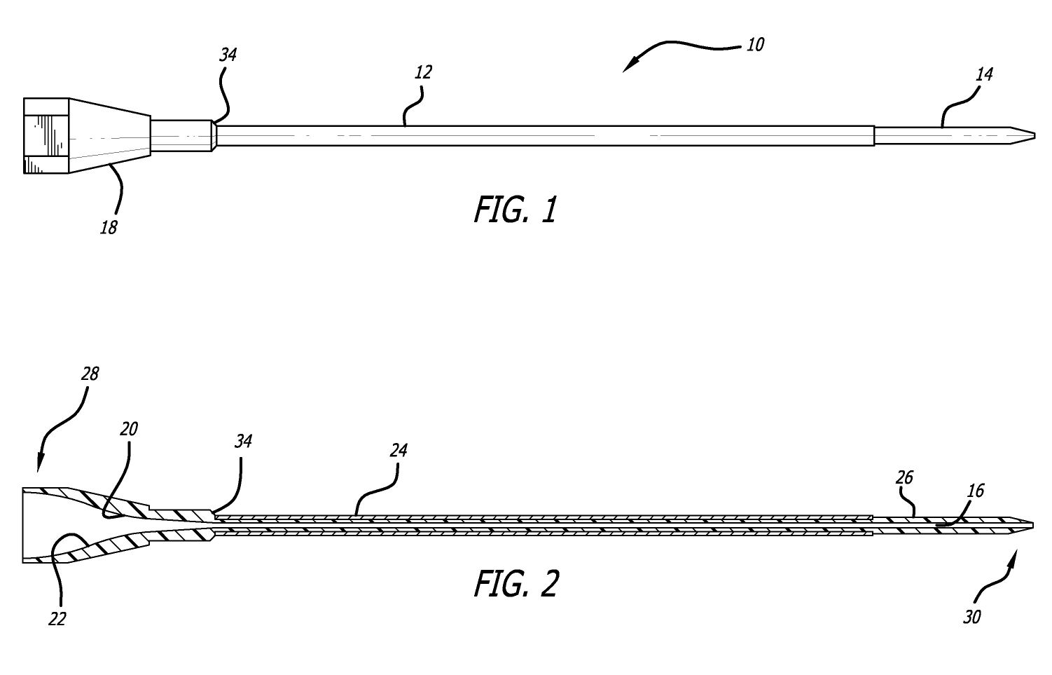

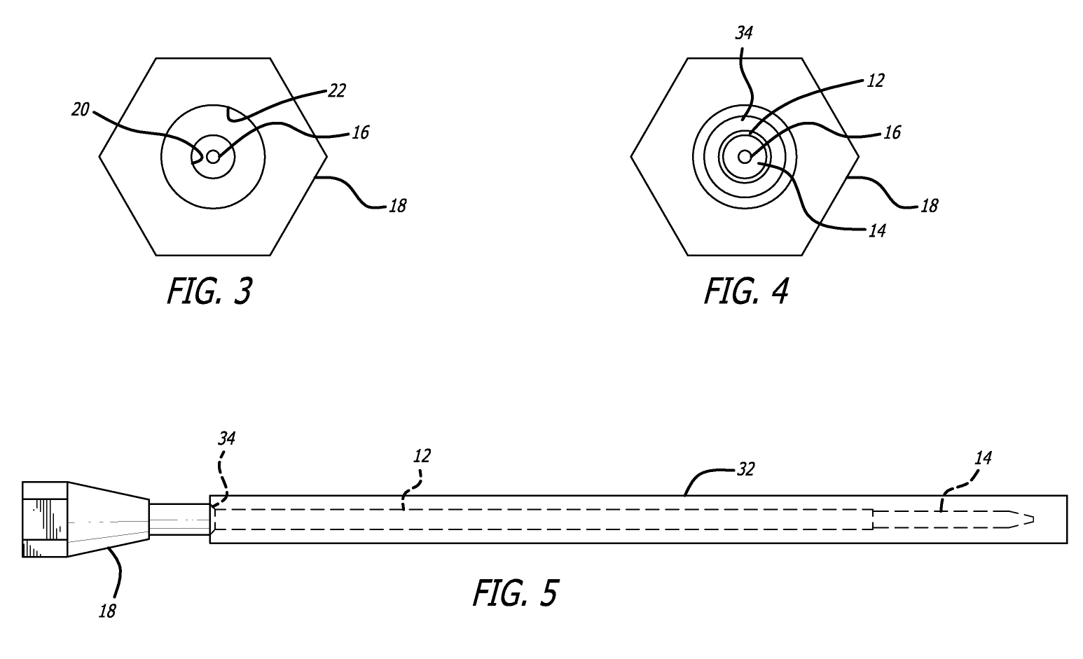

### DETAILED DESCRIPTION OF THE PREFERRED EMBODIMENTS

As is illustrated in the drawings, which are provided for the purposes of illustration and not by way of limitation, the invention is embodied in an improved guidewire introducer and shaping tool 10 that is manufactured with coaxial tubular sheaths including a metal outer layer 12 and a polymer inner layer, such as a plastic liner or polymer tubing shaft 14 having an inner guidewire lumen 16. The introducer may be fabricated similar to a plastic introducer in that a polymer luer hub 18 can be insert-injection molded onto the polymer tubing shaft such that the internal taper 20 of the inner lumen 22 of the hub has a smooth transition to the inner diameter of the lumen of the polymer tubing shaft. The outer metal layer is typically formed by an outer hypotube or cannula 24 that may be laminated, bonded or fitted over the outer surface 26 of the polymer shaft such that the cannula is rigidly attached into the hub and so that the cannula gives increased support and stiffness to the polymer shaft. Alternatively, the metal and polymer layers can be co-extruded. The introducer has a proximal end 28 at the luer hub, and an opposing distal end 30, and the polymer shaft may extend distally beyond the hypotube, because the shaping of the guidewire is typically conducted near the hub. The polymer inner layer or plastic liner is continuous under the outer metal cannula and prevents the end of the metal cannula from contacting a guidewire inserted through or retracted back through the introducer. From this novel construction, a guidewire in the introducer is prevented from contacting the sharp edges of the cannula, but the outer support of the cannula provides the necessary stiffness, and the inner polymer tubing shaft provides a guidewire lumen with a reduced diameter, that together permit adequate shaping of the wire. An outer polymeric tubular sheath 32 may also be removably fitted onto a distal face 34 of the hub and over the coaxial tubular sheaths of the introducer and typically extending beyond the distal end of the introducer to protect the introducer until the introducer is ready to be used.

Two prototypes were manufactured by press-fitting a length of 14 ga hypotubing over an existing Merit plastic introducer, although the thickness of the inner polymer layer may be reduced in view of the metal reinforcement.

It will be apparent from the foregoing that while particular forms of the invention have been illustrated and described, various modifications can be made without departing from the spirit and scope of the invention.

1. A guidewire introducer and shaping tool for placement of guidewires within a patient's vasculature, comprising:
an inner tubular shaft formed of polymeric material having a distal end and a proximal end, said inner tubular shaft having an inner guidewire lumen; and
a tubular metal outer layer surrounding said inner tubular shaft, said tubular metal outer layer having a distal end and a proximal end and extending from said proximal end of said inner tubular shaft over at least a portion of said inner tubular shaft.

2. The guidewire introducer and shaping tool of claim 1, wherein said inner tubular shaft further comprises a luer hub formed of polymeric material at said proximal end of said inner tubular shaft, said luer hub having a hub inner lumen connected with said inner guidewire lumen of said inner tubular shaft.

3. The guidewire introducer and shaping tool of claim 2, wherein said hub inner lumen includes a surface defining an internal taper forming a smooth transition to said inner guidewire lumen of said inner tubular shaft.

4. The guidewire introducer and shaping tool of claim 1, wherein said inner tubular shaft extends distally beyond said distal end of said tubular metal outer layer.

5. The guidewire introducer and shaping tool of claim 2, wherein said proximal end of said tubular metal outer layer is rigidly attached to said luer hub such that said tubular metal outer layer provides support and stiffness to said inner tubular shaft.

6. The guidewire introducer and shaping tool of claim 2, wherein said luer hub has a distal external face, and further comprising an outer polymeric tubular sheath removably fitted onto said distal external face of said luer hub and over said a tubular metal outer layer, said outer polymeric tubular sheath extending beyond the distal ends of said inner tubular shaft and said tubular metal outer layer.

7. A guidewire introducer and shaping tool for placement of guidewires within a patient's vasculature, comprising:
an inner tubular shaft formed of polymeric material having a distal end and a proximal end, said inner tubular shaft having an inner guidewire lumen;
a luer hub formed of polymeric material at said proximal end of said inner tubular shaft, said luer hub having a hub inner lumen connected with said inner guidewire lumen of said inner tubular shaft; and
a tubular metal outer layer surrounding said inner tubular shaft, said tubular metal outer layer having a distal end and a proximal end and extending from said proximal end of said inner tubular shaft over at least a portion of said inner tubular shaft.

8. The guidewire introducer and shaping tool of claim 7, wherein said hub inner lumen includes a surface defining an internal taper forming a smooth transition to said inner guidewire lumen of said inner tubular shaft.

9. The guidewire introducer and shaping tool of claim 7, wherein said inner tubular shaft extends distally beyond said distal end of said tubular metal outer layer.

10. The guidewire introducer and shaping tool of claim 7, wherein said proximal end of said tubular metal outer layer is rigidly attached to said luer hub such that said tubular metal outer layer provides support and stiffness to said inner tubular shaft.

11. The guidewire introducer and shaping tool of claim 7, wherein said luer hub has a distal external face, and further comprising an outer polymeric tubular sheath removably fitted onto said distal external face of said luer hub and over said a tubular metal outer layer, said outer polymeric tubular sheath extending beyond the distal ends of said inner tubular shaft and said tubular metal outer layer.
